(19) United States
(12) Patent Application Publication
(10) Pub. No.: US 2015/0093775 A1
(43) Pub. Date: Apr. 2, 2015

(54) SYSTEM AND METHOD FOR ANALYTE SENSING AND MONITORING

(71) Applicants: Govind RAO, Ellicott City, MD (US); Xudong GE, Ellicott City, MD (US); Yordan KOSTOV, Columbia, MD (US); Madhubanti CHATTERJEE, Baltimore, MD (US); Leah TOLOSA, Columbia, MD (US); Shaunak Dilip UPLEKAR, Cary, NC (US); Manohar PILLI, Gwynn Oak, MD (US)

(72) Inventors: Govind RAO, Ellicott City, MD (US); Xudong GE, Ellicott City, MD (US); Yordan KOSTOV, Columbia, MD (US); Madhubanti CHATTERJEE, Baltimore, MD (US); Leah TOLOSA, Columbia, MD (US); Shaunak Dilip UPLEKAR, Cary, NC (US); Manohar PILLI, Gwynn Oak, MD (US)

(21) Appl. No.: 14/321,904

(22) Filed: Jul. 2, 2014

Related U.S. Application Data
(63) Continuation-in-part of application No. 13/823,903, filed on Jul. 8, 2013.
Application No. 13/823,903 is a continuation-in-part of application No. 13/823,897, now abandoned.

(51) Int. Cl. C12Q 1/04

(52) U.S. Cl. CPC C12Q 1/04
USPC 435/34; 435/287.1

(57) ABSTRACT

An analyte sensing and monitoring system and method is provided that is particularly applicable to monitoring of analytes in cell cultures. The system and method relies on the initial diffusion rates of the analytes from the medium that contains the analytes being measured (e.g., cell culture) into a diffusion chamber that is inserted into the medium, and remote sensing of the analytes using an analyte sensing system that is coupled to the diffusion chamber.

This application is a continuation-in-part of U.S. patent application Ser. No. 13/823,903, filed on Sep. 30, 2011, which claims priority to Provisional Patent Application No. 61/388,170, filed on Sep. 30, 2010. This application is also a continuation-in-part of U.S. patent application Ser. No. 13/823,897, filed on Sep. 30, 2011, which claims priority to Provisional Patent Application No. 61/388,219, filed on Sep. 30, 2010. The disclosures of the above-listed applications are incorporated herein by reference in their entirety.

### BACKGROUND OF THE INVENTION

1. Field of the Invention

The present invention relates to sensing of analytes such as, for example, the sensing of dissolved oxygen, dissolved CO₂ or other type of analyte in a medium.

2. Background of the Related Art

The Background of the Related Art and the Detailed Description of Preferred Embodiments below cite numerous technical references, which are listed in the Appendix below. The numbers shown in brackets at the end of some of the sentences refer to specific references listed in the Appendix. For example, a “[1]” shown at the end of a sentence refers to reference “1” in the Appendix below. All of the references listed in the Appendix below are incorporated by reference herein in their entirety.

Cell culture refers to the process by which prokaryotic or eukaryotic cells are grown in vitro, ideally under controlled environmental conditions. Cell culture technology finds applications in various areas including investigating basic cell biology, relationships between disease causing agents and cells, effects of drugs on cells, genetic engineering and gene therapy. Sensors for accurate real-time measurement of process parameters in cell culture can help to maintain ideal culture conditions. Perturbations in the oxygen supply, pH, or CO₂ in the culture medium can cause unexpected changes in cell metabolism. Oxygen is very commonly measured, as it can become a limiting substrate for cell growth due to its low solubility in water. CO₂, a product of cell respiration, influences the metabolic activity of cells, while pH control determines the optimal cell growth. Hence, the careful monitoring of dissolved oxygen (DO₂), dissolved CO₂ (DCO₂), and pH is very critical. [1]-[5]

Recent developments show the effectiveness of commercial small-scale shaken systems with instrumented controllable micro- and mini-bioreactors where each reactor is designed as a single-use bioreactor. [6]-[8] These systems are often equipped with disposable optical sensors for continuous monitoring or controlling of pH and DO₂. Optical sensors have a lot of advantages over traditional electrochemical sensors, such as high sensitivity, easy miniaturization, free of electromagnetic interference, etc. In addition, the measurement made with patch sensors is only minimally invasive. Except for the patch, which is affixed inside the bioreactor, the measurement is made noninvasively through the transparent vessel wall. These characteristics make them an ideal choice as sensors in these small-scale platforms for process optimization. [2], [9]-[12] However, despite these advantages, there are concerns regarding the effects of the patch on cells. Even though the sensing dye is immobilized in polymer matrix, there is a chance that the dye might leach and cause toxicity. Studies show that the pH and DO₂ patches have no apparent negative effects on the cellular physiology at the transcript level and on the product quality of Hybridoma cell culture. [13] However, the same cannot be predicted for other more sensitive cell lines. Certain cell lines may be too sensitive to allow the use of patch sensors. Some cell lines may require special treatment. For example, for adherent cell lines the patch needs to be treated with protein to make the cells adhere to it. [14] This is often a very tedious task, requiring many steps that need to be implemented carefully.

Thus, there is a need for an alternative system and method for the monitoring of analytes, such as dissolved O₂ and dissolved CO₂ in a liquid medium.

### SUMMARY OF THE INVENTION

An object of the invention is to solve at least the above problems and/or disadvantages and to provide at least the advantages described hereinafter.

Therefore, an object of the present invention is to provide a system and method for the sensing of analytes.

Another object of the present invention is to provide a system and method for the sensing of gaseous analytes dissolved in a liquid medium.

Another object of the present invention is to provide a system and method for the sensing of dissolved O₂ in a liquid medium.

Another object of the present invention is to provide a system and method for the sensing of dissolved CO₂ in a liquid medium.

Another object of the present invention is to provide a system and method for the sensing of dissolved O₂ and CO₂ in a cell culture.

To achieve at least the above objects, in whole or in part, there is provided a system for measuring at least one analyte present in a medium, comprising a vessel adapted to a contain the medium, a diffusion chamber positioned inside the vessel, wherein the diffusion chamber comprises at least one wall that defines a diffusion chamber volume and that is at least partially permeable to the at least one analyte, an analyte sensing system coupled to the diffusion chamber for detecting portions of the at least one analyte that diffuse into the diffusion chamber from the medium and a controller operatively coupled to the analyte sensing system that determines initial rates of diffusion of the at least one analyte from the medium into the diffusion chamber based on amounts of the at least one analyte detected by the analyte sensing system, and for further determining a concentration of the at least one analyte in the medium based on the initial rates of diffusion.

To achieve at least the above objects, in whole or in part, there is also provided a system for measuring O₂ and CO₂ in a cell culture, comprising a vessel adapted to a contain the cell culture, a diffusion chamber positioned inside the vessel, wherein the diffusion chamber comprises at least one wall that defines a diffusion chamber volume and that is at least partially permeable to O₂ and CO₂, an O₂ analyzer and a CO₂ analyzer pneumatically coupled to the diffusion chamber for detecting O₂ and CO₂ that diffuse into the diffusion chamber from the cell culture and a controller operatively coupled to the O₂ analyzer and CO₂ analyzer that determines initial rates of diffusion of O₂ and CO₂ from the cell culture into the diffusion chamber volume based on the O₂ and CO₂ detected by the O₂ analyzer and CO₂ analyzer, and for further determining concentrations of O₂ and CO₂ in the cell culture based on the initial rates of diffusion.

To achieve at least the above objects, in whole or in part, there is also provided a method for measuring at least one analyte present in a medium, comprising positioning a diffusion chamber within the medium, wherein the diffusion chamber comprises at least one wall that defines a diffusion chamber volume and that is at least partially permeable to the at least one analyte, flushing the diffusion chamber to substantially remove the at least one analyte from the diffusion chamber, allowing the at least one analyte to diffuse from the medium into the diffusion chamber, detecting portions of the at least one analyte that have diffused into the diffusion chamber, determining initial rates of diffusion of the at least one analyte from the medium into the diffusion chamber based on the detected portions of the at least one analyte and determining a concentration of the at least one analyte in the medium based on the determined initial rates of diffusion.

To achieve at least the above objects, in whole or in part, there is also provided a probe system for measuring at least one analyte present in a medium, comprising a housing defined by housing walls that are impermeably to the analyte being measured, a predetermined length of tubing that is permeable to the analyte being measured contained in the housing, wherein two open ends of the tubing extend through openings in a first end of the housing so as to be positioned outside the housing, and a sampling portion of the tubing extends through openings in a second end of the housing so as to be positioned outside the housing, wherein the two open ends of the tubing are coupled to an analyte sensing system for detecting portions of the at least one analyte that diffuse into the sampling portion of the tubing from the medium when the sampling portion of the tubing is in contact with the medium and a controller operatively coupled to the analyte sensing system that determines initial rates of diffusion of the at least one analyte from the medium into the sampling portion of the tubing based on amounts of the at least one analyte detected by the analyte sensing system, and for further determining a concentration of the at least one analyte in the medium based on the initial rates of diffusion.

To achieve at least the above objects, in whole or in part, there is also provided a probe for measuring at least one analyte present in a medium, comprising a housing defined by housing walls that are impermeably to the analyte being measured and a predetermined length of tubing that is permeable to the analyte being measured contained in the housing, wherein two open ends of the tubing extend through openings in a first end of the housing so as to be positioned outside the housing, and a sampling portion of the tubing extends through openings in a second end of the housing so as to be positioned outside the housing, wherein the two open ends of the tubing are adapted to be coupled to an analyte sensing system for detecting portions of the at least one analyte that diffuse into the sampling portion of the tubing from the medium when the sampling portion of the tubing is in contact with the medium.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1A is a block diagram that illustrates the principle of operation of one preferred embodiment of the present invention;

FIG. 1B is a block diagram that illustrates a probe system, in accordance with one embodiment of the present invention;

FIG. 2 is schematic diagram of a system for sensing two gas analytes, in accordance with one embodiment of the present invention;

FIG. 3 is schematic diagram of a system for sensing two gas analytes that utilizes a T-flask as the diffusion chamber, in accordance with one embodiment of the present invention;

FIG. 4 is schematic diagram of a system for sensing two gas analytes that utilizes a spinner-flask as the diffusion chamber, in accordance with one embodiment of the present invention;

FIG. 5 is a flowchart of steps in the operation of the systems of FIGS. 1-4, in accordance with one preferred embodiment of the present invention;

FIG. 6 is a plot that shows a typical O₂ and CO₂ concentration profile in the diffusion chamber during measurement;

FIG. 7 is a plot showing the correlation of the initial diffusion rate of O₂ into the diffusion chamber versus the feed O₂ concentration as measured by a DO₂ patch;

FIG. 8 is a plot showing the correlation of the initial diffusion rate of CO₂ into the diffusion chamber versus the feed CO₂ concentration as measured by a DCO₂ patch;

FIG. 9A is a plot showing the DO₂ profile measured by the diffusion rate-based method of the present invention, as well as the DO₂ profile measured by a DO₂ patch sensor, using the system of FIG. 3 (T-flask);

FIG. 9B is a plot showing the DO₂ profile measured by the diffusion rate-based method of the present invention, as well as the DO₂ profile measured by a DO₂ patch sensor, using the system of FIG. 4 (spinner-flask);

FIG. 10A is a plot showing the DCO₂ profile measured by the diffusion rate-based method of the present invention, as well as the DCO₂ profile measured by a DCO₂ patch sensor, using the system of FIG. 3 (T-flask); and

FIG. 10B is a plot showing the DCO₂ profile measured by the diffusion rate-based method of the present invention, as well as the DCO₂ profile measured by a DCO₂ patch sensor, using the system of FIG. 4 (spinner-flask).

### DETAILED DESCRIPTION OF PREFERRED EMBODIMENTS

Certain terms are used throughout the following description and claims to refer to particular system components. As one skilled in the art will appreciate, companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . . ” Also, the term “couple” or “coupled” is intended to mean either a direct or indirect connection, or through an indirect connection via other devices and connections.

By way of example, the present invention will be predominantly described in connection with a system and method for the sensing of dissolved analytes in a liquid medium, that is particularly suited for the sensing and monitoring of dissolved O₂ (“DO₂”) and dissolved CO₂ (“DCO₂”) in a cell culture. However, it should be appreciated that the present invention can be used for the sensing and monitoring of any type of analyte that can diffuse through any type of permeable boundary.

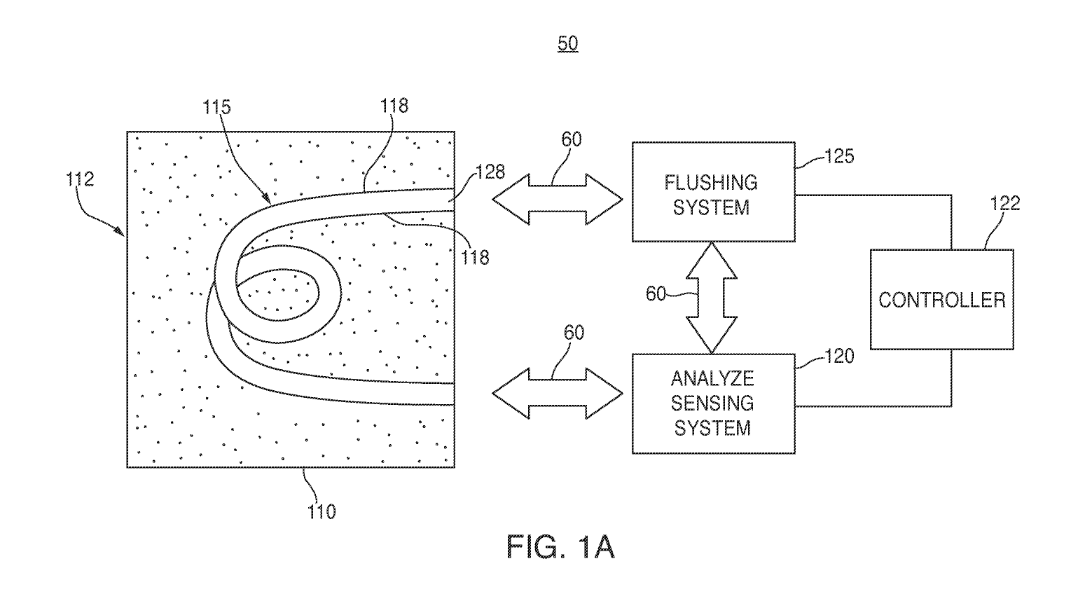

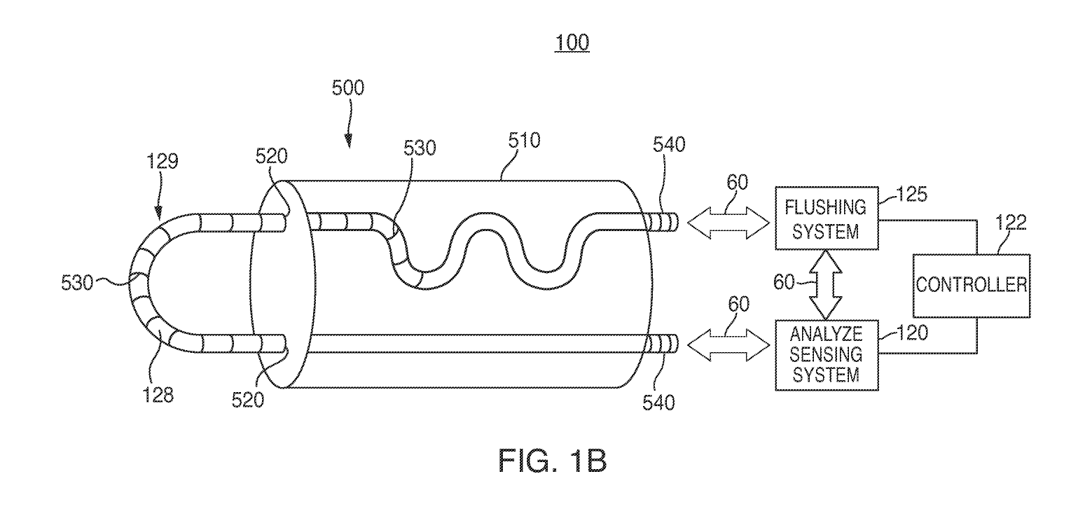

FIG. 1A is a block diagram that illustrates the principle of operation of one preferred embodiment of the present invention. The system 50 includes a diffusion chamber 115 that is adapted to be positioned inside a medium 112 that contains the analyte(s) to be measured. In the embodiment of FIG. 1, the medium 112 is contained in a vessel 110. The system 50 also includes an analyte sensing system 120, a flushing system 125 and a controller 122. The diffusion chamber 115 has at least one diffusion chamber wall 118 that is permeable to the analyte(s) being measured so that it functions as a diffusion membrane between the medium 112 and the volume defined by the diffusion chamber wall(s) 118.

The flushing system 125 is provided for initializing the system 50 prior to performing a measurement by flushing out (removing) any residual amounts of the analyte(s) to be measured from the diffusion chamber 115 and analyte sensing system 120. The flushing system 125 suitably utilizes nitrogen as the flushing agent.

The diffusion chamber 115, analyte sensing system 120 and flushing system 125 are coupled via coupling components 60, which are preferably pneumatic coupling components. The coupling components 60 are suitably any combination of components such as, for example, tubing, valves, conduits, pumps, intake ports, exhaust ports, etc.

The diffusion chamber is preferably flexible tubing 128 that is permeable to the analyte(s) being measured, however any other type of diffusion chamber 115 with at least one wall that is permeable to the analyte being measured can be used. The flexible tubing 128 is suitably silicone tubing, but it can be made of any material that is permeable to the analyte(s) being measured. The more flexible tubing 128 that is present in the medium 112, the more analyte diffusion through the walls 118 of the tubing 128 due to the larger tubing surface area in contact with the medium 112. However, the total volume of the flexible tubing 128 that is present in the medium 112 is preferably adjusted so that a sufficient amount of analyte diffuses through the walls 118 for measurement by the analyte sensing system 120, but not so much that it adversely affects the analyte concentrations in the medium 112.

The coupling components 60 are impermeable to the analyte(s) being measured so that the analytes do not diffuse out of the coupling components 60 on the way to the analyte sensing system 120. For example, if tubing is used as some of the coupling components 60, the tubing should be impermeable to the analyte(s) being measured.

The vessel 110 can be any container or vessel that is capable of holding the medium 112. For example, the vessel 110 can be a container for holding a cell culture such as, for example, a T-flask or a spinner-flask, as will be discussed in more detail below. If the vessel 110 is a container for holding a cell culture and one of the analytes being measured is O₂, the size and/or length of the tubing 128 should be set so that a sufficient amount of O₂ diffuses into the tubing for measurement by the analyte sensing system 120, while not taking away so much O₂ from the cell culture that cell growth in inhibited.

The analyte sensing system 120 can include any combination of sensors/analyzers known in the art for detecting the analyte(s) being measured. For example, if the analytes being measured are O₂ and CO₂, then the analyte sensing system 120 suitably includes an O₂ analyzer and a CO₂ analyzer.

The controller 122 is operatively coupled to the analyte sensing system 120, the flushing system and optionally one or more coupling components (e.g., pumps, valves, etc.) so as to control the measurement process and analyze data from the analyte sensing system 120, as will be described in more detail below.

FIG. 1B is a block diagram that illustrates a probe system 100, in accordance with one embodiment of the present invention. The probe system 100 is similar to the system 50 of FIG. 1A, except that the diffusion chamber 115 forms part of a probe 500 that can be deployed in a variety of applications requiring the measurement of analytes. For example, probe 500 can be inserted into soil, a liquid, a gas environment or the human body to measure predetermined analytes.

As in the system 50 of FIG. 1A, the diffusion chamber 115 is preferably flexible tubing 128. The probe housing 510 is made of a material that is impermeable to the analyte being measured. This is suitably a plastic that is impermeable to the analyte being measured, but it can be any material that is impermeable to the analyte being measured. The portion of the tubing 128 that extends outside of the probe housing 510 is hereinafter referred to as the “sampling portion” 129 (equivalent to the diffusion chamber 115).

As discussed above, the more tubing 128 that is exposed to the analyte being measured, the more of the analyte will diffuse into the tubing 128 due to the larger tubing surface area in contact with the analyte being measured. Thus, the sensitivity of the probe system 100 can be adjusted by adjusting the amount of tubing 128 that is present outside the probe housing 510, thereby increasing the surface area of the sampling portion 129. However, as discussed above, the total volume of the sampling portion 129 is preferably adjusted so that a sufficient amount of analyte diffuses through the walls of the tubing 128 for measurement by the analyte sensing system 120, but not so much that it adversely affects the analyte concentrations in the medium that contains the analyte.

To this end, a sufficient amount of “slack” in the tubing 128 is provided inside the probe housing 510 such that the amount of tubing 128 extending outside of the probe housing 510 can be adjusted depending on the analyte sensitivity required. For example, if more analyte sensitivity is required, then more tubing 128 can be pulled from the probe housing 510 so that more tubing 128 is exposed to the analyte being measured, resulting in larger sampling portion 129. If less analyte sensitivity is desired, then some of the tubing 128 can be pushed back in to the probe housing 510, resulting in a smaller sampling portion 129. O-rings 520 are preferably used to provide a seal that keeps the analyte being measured from entering the probe housing 510, while allowing the tubing 128 to be selectively pulled out of or pushed into the probe housing 510.

The probe system 100 is preferably calibrated so that it is known how the sensitivity of the probe system 100 varies as a function of how much tubing 128 is present outside the probe housing 510. Further, markings 530 can be optionally placed on the tubing 128 so that a user can use the markings 530, in conjunction with the calibration information, to determine how much tubing 129 should be pulled out of the probe housing 510 for any particular analyte sensing application.

The ends 540 of the tubing 128 are preferably barbed so as to easily connect to coupling components 60. The coupling components 60, flushing system 125, analyte sensing system 120 and controller 122 work as described above in connection with the system 50 of FIG. 1A.

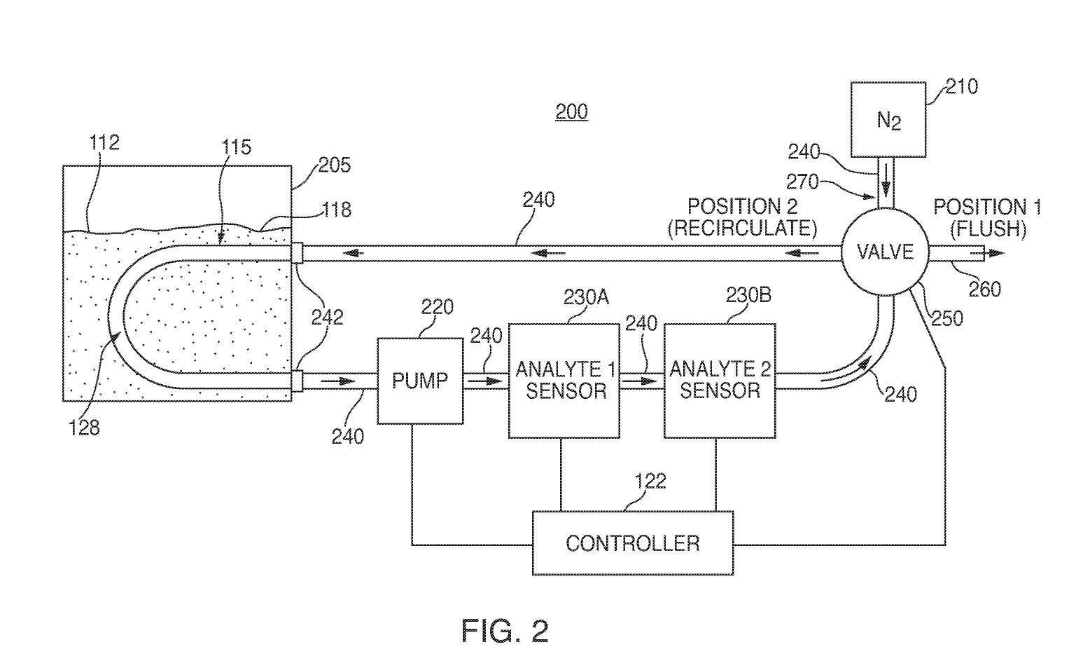

FIG. 2 is schematic diagram of a system for sensing two gas analytes, in accordance with one embodiment of the present invention. The system 100 includes a diffusion chamber 115, a sensor for a first analyte 230A, a sensor for a second analyte 230B, a pump 220, a three-way valve 250, a flushing gas source 210 and a controller 122. A vessel 205 is used for holding a medium 112 that contains the analytes being measured. Pump 220 is preferably a pneumatic pump.

The diffusion chamber 115 is preferably flexible tubing 128 made of silicone. The tubing 128, analyte sensors 230A and 230B, pump 220 and valve 250 are coupled together with conduits 240, thereby forming a circulation loop through which gases can circulate. The conduits 240 can be any conduit capable of circulating air/gas and that is impermeable to the analytes being measured, such as, for example, Tygon® tubing. The conduits 240 are coupled to ends of the tubing 128 via couplers 242, which are suitably standard connectors, such as hose barbs or luer lock fittings.

The medium 112 in which the analyte being measured is contained is placed inside the vessel 205. The medium 112 could be, for example, a liquid medium (e.g., a cell culture) in which the analytes being measured are in a dissolved state. The flushing source 210 is preferably nitrogen, but any other inert gas can be used as the flushing source 210.

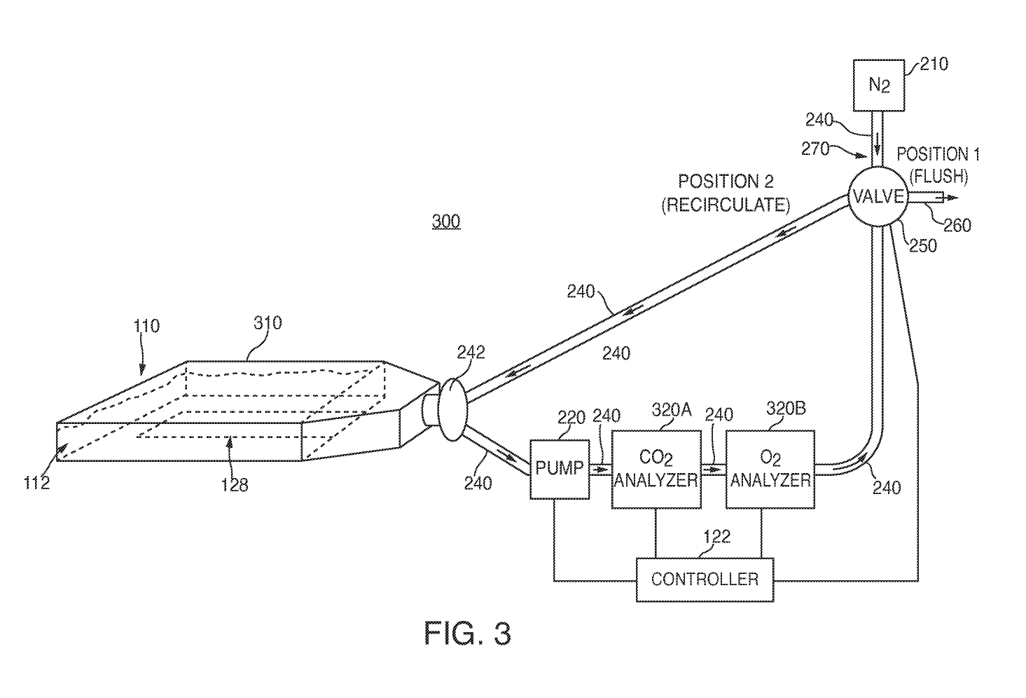

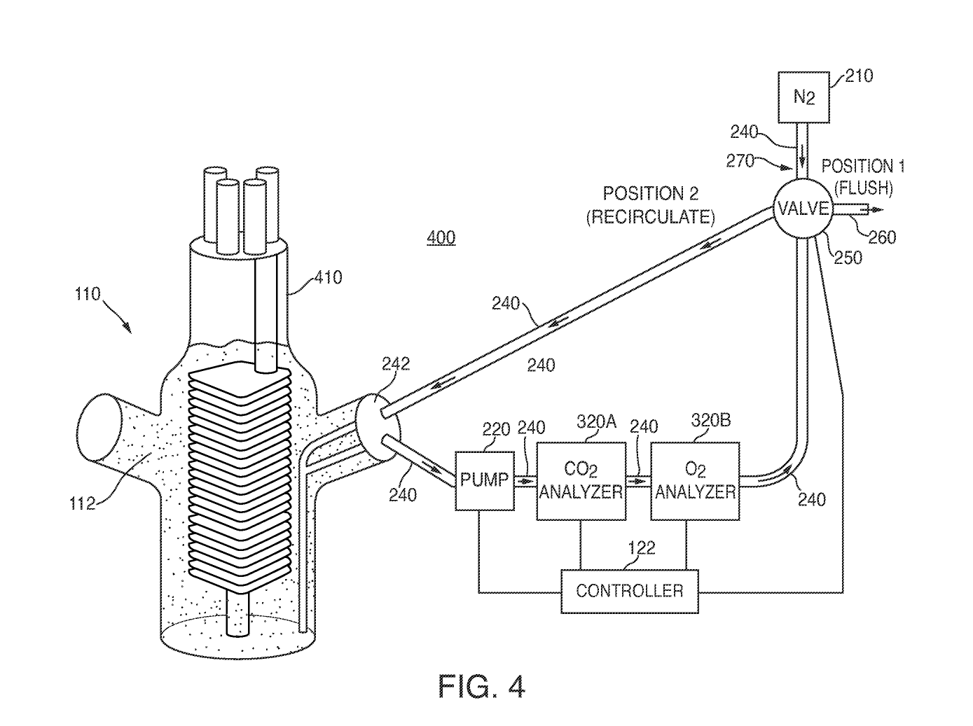

FIGS. 3 and 4 are schematic diagrams of systems 300 and 400, respectively, similar to the system 200 of FIG. 2, but adapted to measure O₂ and CO₂ present in a cell culture, in accordance with other embodiments of the present invention. The systems 300 and 400 of FIGS. 3 and 4 are substantially the same, except for the type of vessel 110 used to contain the medium 112. The system 300 of FIG. 3 utilizes a T-flask 310 as the vessel 110, whereas the system 400 of FIG. 4 utilizes a spinner-flask 410. Both systems 300 and 400 utilize coupler 242 for coupling the ends of the tubing 128 to the conduits 240. In system 300, the tubing 128 in the T-flask 310 is suitably silicone tubing having an external diameter of approximately 3 mm, a wall thickness of approximately 0.5 mm and a length of approximately 10 cm. In system 400, the tubing 128 in the spinner-flask 410 is preferably silicone tubing having an external diameter of approximately 3 mm, a wall thickness of approximately 0.5 mm and a length of approximately 5 cm.

In systems 300 and 400, the medium 112 is a cell culture. However, any type of medium that contains analytes to be measured can be used as the medium 112. The analyte sensors in systems 300 and 400 are a CO₂ analyzer 320A and an O₂ analyzer 320B. A suitable CO₂ analyzer is an infrared CO₂ analyzer. A suitable O₂ analyzer is a polarographic oxygen analyzer.

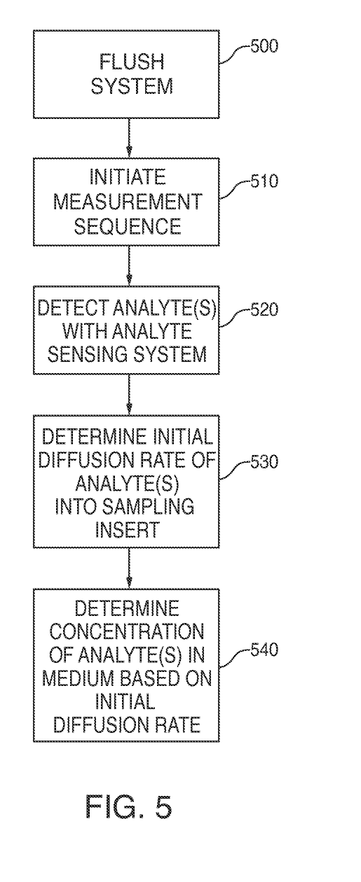

FIG. 5 is a flowchart of steps in the operation of systems 50, 100, 200, 300 and 400, in accordance with one preferred embodiment of the present invention. The process begins at step 500, in which the tubing 128, the analyte sensing system 120 and the coupling components 60 are flushed with flushing system 125, preferably using nitrogen as the flushing agent. This flushing procedure removes any of the analytes being measured from the diffusion chamber 115, analyte sensing system 120 and coupling components 60.

The controller 122 controls the flushing and measurement processes, including control of the pump 220 and valve 250. In addition, the controller 122 receives measurement data from the analyte sensors 230A, 230B, 320A and 320B. When a measurement is ready to be made, the controller 122 will initiate a flush sequence at step 500, which will place three-way valve 250 into position 1. This will open flushing inlet 270 and exhaust port 260 in three-way valve 250. The controller 122 also actuates pump 220, thereby drawing nitrogen into conduits 240 via flushing inlet 270, passing the nitrogen through the diffusion chamber 115, pump 220, analyte sensors (230A, 230B, 320A, 320B) and out of exhaust port 260. This flush sequence is preferably performed until the analyte sensors (230A, 230B, 320A, 320B) output a zero reading (after accounting for instrument offsets). This helps to achieve constant initial conditions for all measurements.

Once the flush sequence is complete, a measurement sequence is initiated at step 510. For the measurement sequence, the controller 150 places three-way valve 142 in position 2, which closes off flushing inlet 270 and exhaust port 260 in three-way valve 250, and will actuate the pump 220. This will circulate the remaining nitrogen in the system in a continuous loop through the diffusion chamber 115, pump 220, analyte sensors (230A, 230B, 320A, 320B) and valve 250.

Analytes present in the medium 112 diffuse into the diffusion chamber 115, and are transported to the analyte sensors (230A, 230B, 320A, 320B). The analyte sensors (230A, 230B, 320A, 320B) measure the amount of analytes present at step 520. In systems 300 and 400, the CO₂ analyzer 320A and the O₂ analyzer 320B measure the amounts of CO₂ and O₂ present, respectively.

Then, at step 530, the controller 122 determines an initial rate of diffusion of the analytes into the diffusion chamber 115 based on analyte measurement data received from the analyte sensors (230A, 230B, 320A, 320B). In systems 300 and 400, the controller 122 determines an initial rate of diffusion of CO₂ and O₂ into the diffusion chamber 115 based on measurement data received from the CO₂ analyzer 320A and the O₂ analyzer 320B.

At step 540, the controller 122 determines the concentration of the analytes in the medium 112 based on the initial diffusion rate determined at step 530. In systems 300 and 400, the controller 122 determines the concentrations of CO₂ and O₂ in the medium 112 based on the initial diffusion rate determined at step 530.

The controller 122 is preferably implemented with one or more programs or applications run by one or multiple processors. The programs or applications are respective sets of computer readable instructions stored in a tangible medium that are executed by one or multiple processors.

The processor(s) can be implemented with any type of processing device, such as a general purpose computer, a special purpose computer, a distributed computing platform located in a “cloud”, a server, a tablet computer, a smartphone, a programmed microprocessor or microcontroller and peripheral integrated circuit elements, ASICs or other integrated circuits, hardwired electronic or logic circuits such as discrete element circuits, programmable logic devices such as FPGA, PLD, PLA or PAL or the like. In general, any device on which a finite state machine capable of running the programs and/or applications used to implement the operational steps described above can be used as the processor(s).

### Calculation of Initial Diffusion Rate

The mass balance equation for the whole recirculation system including the diffusion chamber 115, the inside volumes of the pump 220, the analyte sensors (230A, 230B, 320A, 320B) and the conduits 240 can be written as follows

$$V \frac{d C}{d t} = kA ( {C}_{g} - C ) \qquad ( 1 )$$

where V is the total volume of the system, C is the concentration of O₂ or CO₂ in the diffusion chamber 115, t is time, k is the mass transfer coefficient, A is the total mass transfer area, and C_{g} is the concentration of O₂ or CO₂ in the medium 112.

At the beginning of the recirculation (t=0), the O₂ and CO₂ concentration in the diffusion chamber 115 is zero. Thus,

$$\begin{matrix}{C}_{g} = \frac{V}{kA} \frac{d C}{d t} {\vert}_{t = 0} \\ = a \frac{d C}{d t} {\vert}_{t = 0}\end{matrix} \qquad ( 2 )$$

where a=V/kA. From the above equation, it can be seen that the O₂ and CO₂ concentration in the medium 112 is linearly proportional to their initial diffusion rate through the diffusion chamber 115. By monitoring the O₂ and CO₂ concentration in the diffusion chamber 115 and calculating the initial diffusion rate, their concentration in the medium can be determined. The initial diffusion rate can be calculated by fitting the gas concentration trend line in the first few minutes to a linear equation.

### System Tests

Systems 300 and 400 were tested using a cell line is a non-adherent SP 2/0-based myeloma/mouse (2055.5) secreting IgG3 antibody specific for the Neisseria meningitides capsular-polysaccharide (MCPS). One liter of CD Hybridoma GTTM (Invitrogen, Carlsbad, Calif.) stock media solution supplemented with 8 mM L-glutamine (GIBCO) and 2×10⁻⁴% β-mercaptoethanol (v/v) (Sigma, St. Louis, Mo.) was prepared and stored at 4° C.

For testing system 300, 50 ml of this media was added to a re-closable T-flask 310 (polystyrene, growth area 115 cm², max volume 100 ml, TPP, St. Louis, Mo. 63088) and inoculated at an initial cell concentration of 0.2×10⁶ cells/ml. For testing the system 400, 825 ml of the media was added to a spinner-flask 410 and inoculated at an initial cell concentration of 0.25×10⁶ cells/ml.

The cell cultures were monitored in an incubator (not shown) at 37° C. and 5% CO₂ for 1 week. For testing purposes, DO₂ and DCO₂ were measured by patch sensors placed inside the T-flask 310 and spinner-flask 410 (not shown) every 5 minutes and the data was stored. The diffusion rate-based measurement was made every 3 hours by flushing the system with N₂ and switching the 3-way valve 250 to recirculation mode (position 2).

The diffusion rate-based measurement method was calibrated at 37° C. by bubbling the gas mixture through the cell culture media 112. The gas mixtures with desired O₂ and CO₂ concentrations were obtained by mixing pure N₂ and air/CO₂ through two flowmeters (FM4332 and FM4333, Advanced Specialty Gas Equipment Corp., South Plainfield, N.J.). After the gas mixture reached equilibrium with the media 112, the O₂ and CO₂ diffusion rates across the diffusion chamber 115 was measured following the procedures described above. The diffusion rate was measured at 0.0%, 25.0%, 50.0%, 75.0%, and 100.0% O₂ (air saturation) in the feed, and 0.0%, 3.0%, 5.0%, 10.0%, and 20.0% for CO₂.

As discussed above, the diffusion rate-based measurement method of the present invention measures the concentrations of DO₂ and DCO₂ in a medium 112 (e.g., a cell culture) by measuring their initial diffusion rates across the wall(s) of a diffusion chamber 115 immersed in the media 112. Since each measurement starts with flushing the system with N₂ to remove the O₂ and CO₂ originally present in the system, the O₂ and CO₂ concentration in the diffusion chamber 115 is zero before the recirculation begins.

During recirculation, O₂ and CO₂ diffuses across the wall(s) of the diffusion chamber 115 and their concentrations in the system increase with time. If allowed enough time, the O₂ and CO₂ concentrations in the diffusion chamber 115 will eventually reach equilibrium with the DO₂ and DCO₂ in the media 112.

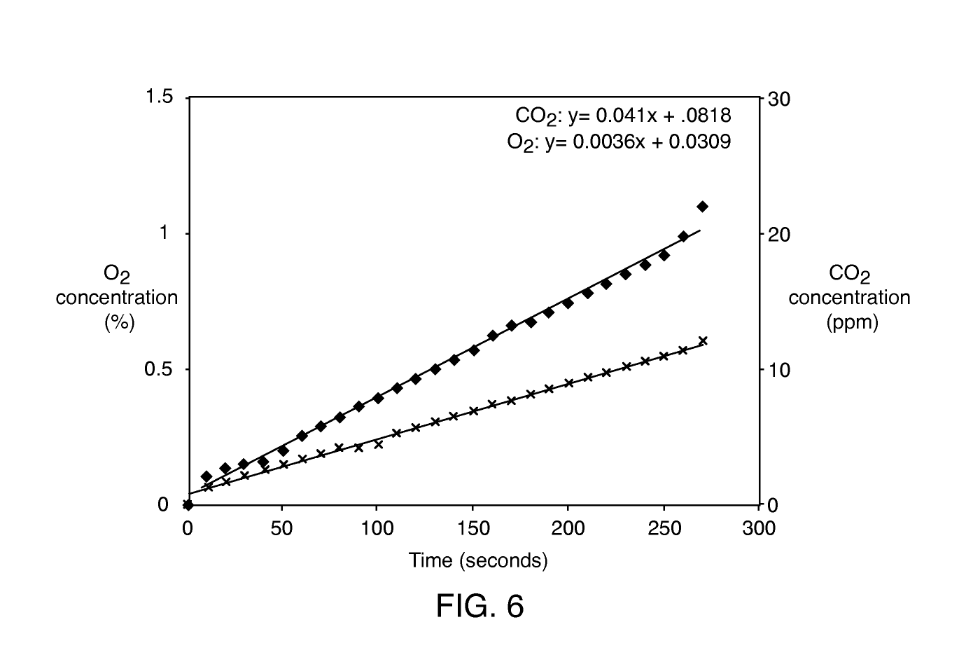

FIG. 6 is a plot that shows a typical O₂ and CO₂ concentration profile in the diffusion chamber 115 during measurement. It can be seen that the O₂ and CO₂ concentration in the diffusion chamber 115 increases linearly with time in the first few minutes after starting the recirculation. By fitting the concentration profile to a linear equation, the initial diffusion rates can be obtained.

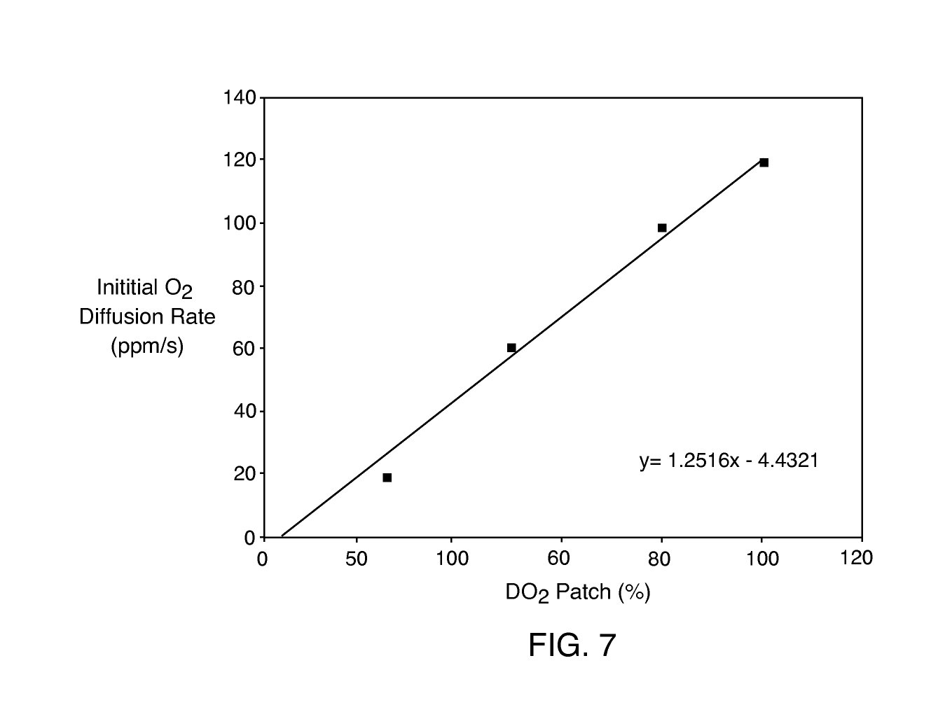

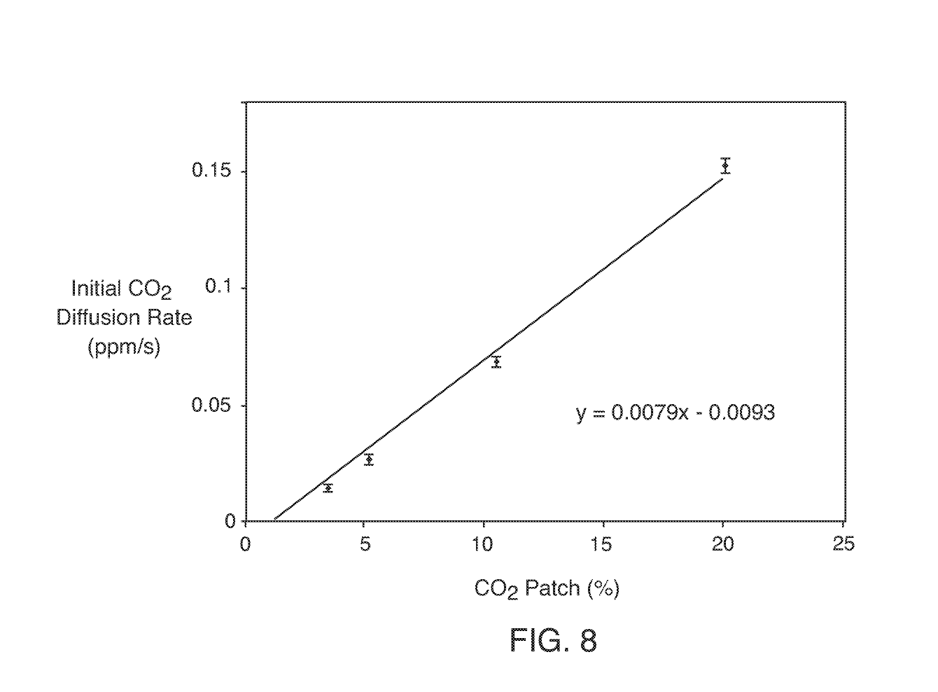

FIGS. 7 and 8 are plots that show the correlation of the initial diffusion rates into the diffusion chamber 115 vs. the feed concentrations (as measured by the DO₂ and CO₂ sensor patches in the T-flask 310 and spinner-flask 410). It can be seen that the O₂ and CO₂ concentration in the feed is linearly proportional to the initial diffusion rate, which is in agreement with equation (2). The slope of the line (1/a) is determined by the volume of the system, the surface area of the diffusion chamber 115 and the mass transfer resistance of the diffusion chamber 115 to the analyte. For a given system, the slope of the line is a constant at a constant temperature. Thus, by measuring the initial O₂ and CO₂ diffusion rates into the diffusion chamber 115, the O₂ and CO₂ level in the media 112 can be obtained.

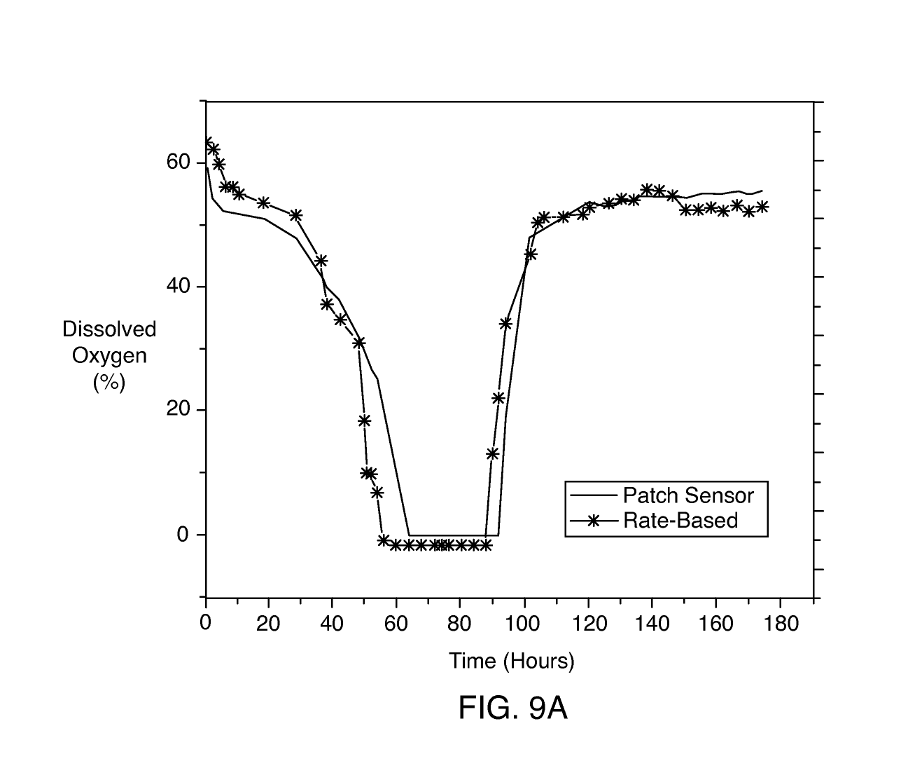

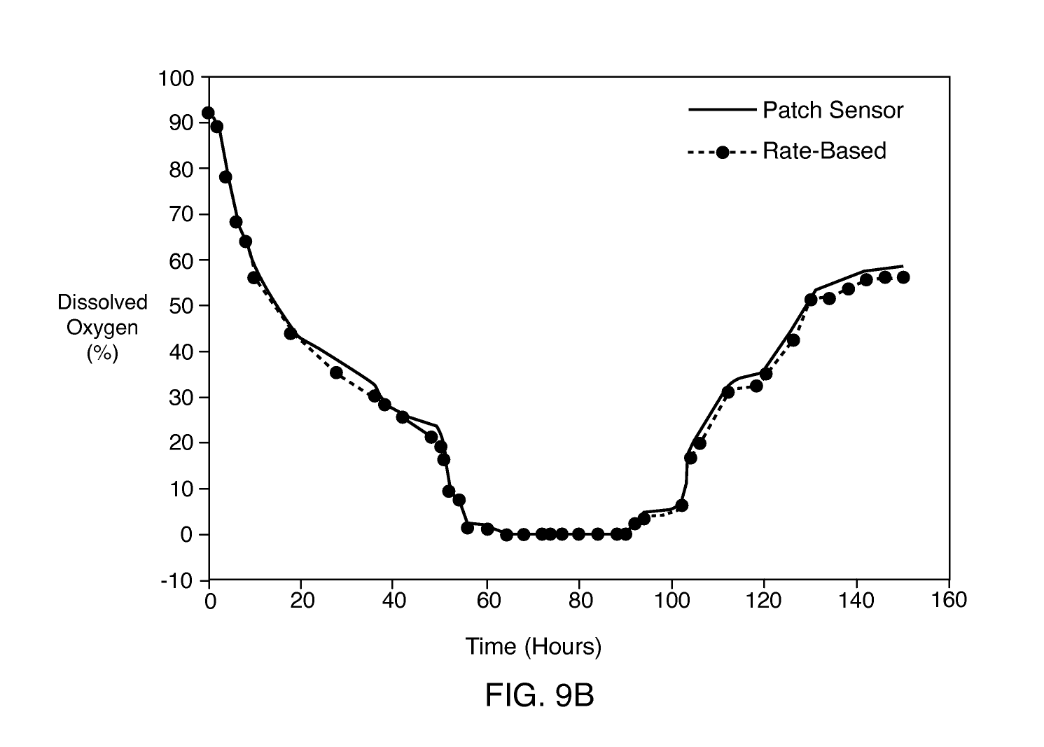

After calibration, the systems 300 and 400 were tested with mammalian cell cultures conducted in a T-flask (system 300) and a spinner-flask (system 400) at 37° C. The cultures were monitored for 7 days. FIGS. 9A and 9B are plots showing the DO₂ profile measured by the diffusion rate-based method of the present invention, as well as by DO₂ patch sensors (not shown) placed inside the T-flask 310 and spinner-flask 410. FIG. 9A is the plot for the T-flask (system 300) and FIG. 9B is the plot for the spinner-flask (system 400).

At the early stage of the cell culture, the cells were growing and consumed more and more O₂. Both methods showed the gradual decrease in DO₂ concentration in the culture. At a late stage of the culture, the cells began to die and consumed less and less O₂. Just as both methods show, the DO₂ concentration in the culture gradually increased in this stage.

O₂ limitation is a common phenomenon in microbial cell cultures even with vigorous shaking. Although mammalian cells do not grow as fast as microbial cells and consume less O₂, the process still became O₂-limited between 60-90 hours for the T-flask 310 and between 58-102 hours for the spinner-flask 410 due to the low solubility of O₂. Despite he use of slow stirring, the O₂-limiting condition lasted longer in the spinner-flask 410 due to the smaller specific mass transfer area for oxygen.

Throughout the process, the measurements obtained using the diffusion rate-based method of the present invention followed the measurements obtained with the patch sensor quite well. The readings from the two different methods were more consistent for the spinner-flask 410, as the media 112 was more uniform due to slow stirring.

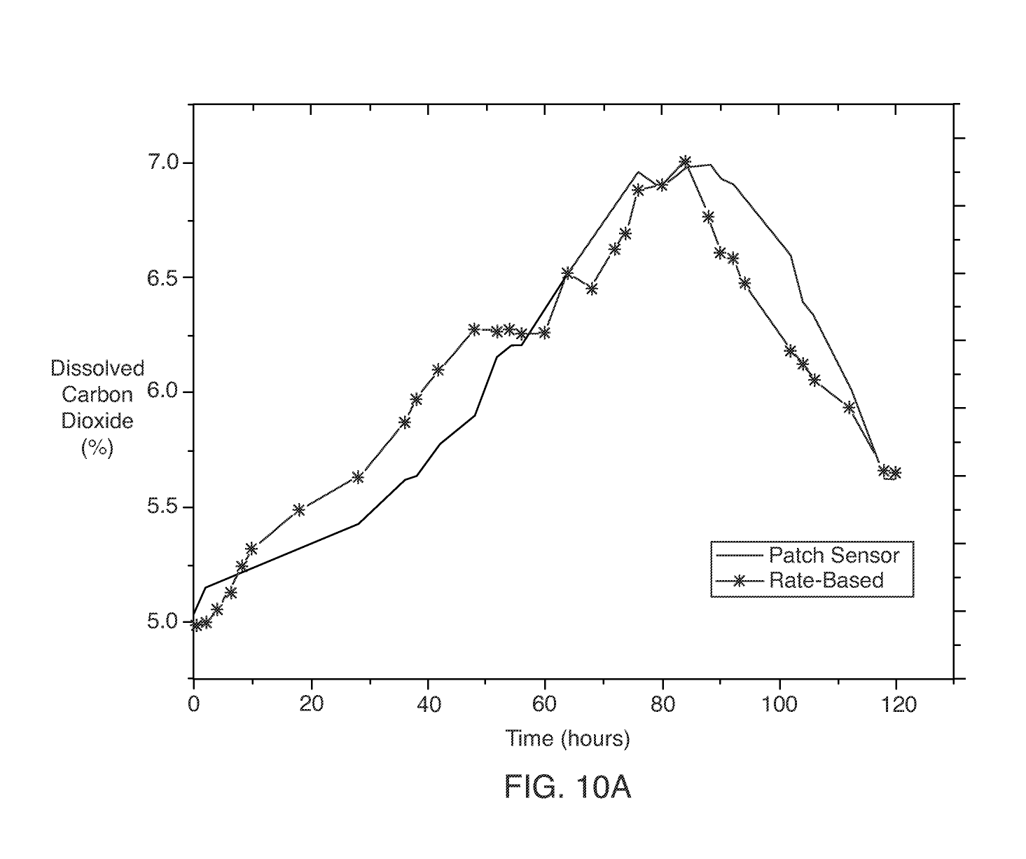

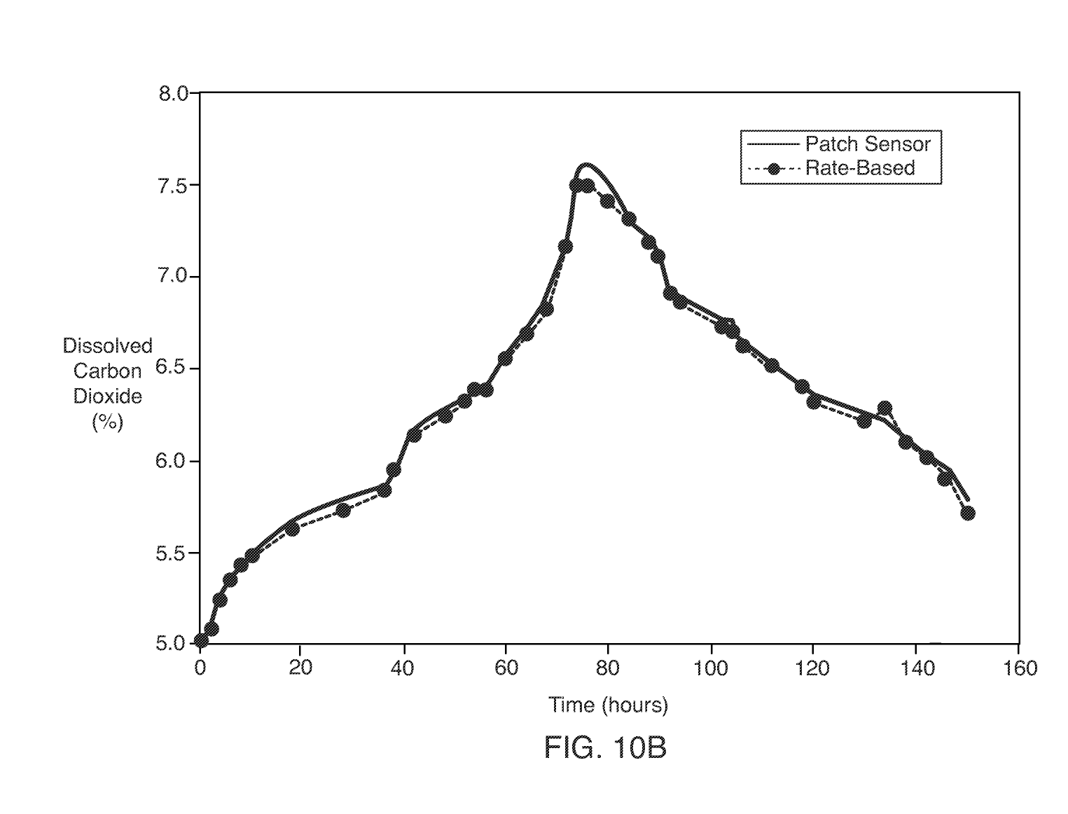

FIGS. 10A and 10B are plots showing the DCO₂ profile measured by the diffusion rate-based method of the present invention, as well as by DCO₂ patch sensors (not shown) placed inside the T-flask 310 and spinner-flask 410. FIG. 10A is the plot for the T-flask (system 300) and FIG. 10B is the plot for the spinner-flask (system 400). As expected, both sensing methods showed a gradual increase in DCO₂ concentration during the first stage of the culture. After the O₂-limiting stage, the cells began to die and produce less and less CO₂. As a result, the DCO₂ concentration in the culture began to gradually decrease. Just as in the DO₂ sensor, the measurements obtained using the diffusion rate-based method of the present invention followed the measurements obtained with the patch sensor quite well. These results show that cell culture processes can be successfully monitored by the diffusion rate-based systems and method of the present invention.

An advantage of the diffusion rate-based systems and methods of the present invention is that there is no direct sensor-media contact, except for the biocompatible diffusion chamber 115 (preferably silicone tubing), which was found to have no effect on the cells. Electrochemical probes are not commonly used in small-scale bioreactors, mainly due to their invasive nature. Patch sensors have some advantages over traditional electrochemical probes in that they are usually disposable and only partially invasive. However, there are still concerns regarding the effect of the patch sensors on the cells, especially extremely sensitive cells.

The diffusion rate-based systems and methods of the present invention do not require reaching mass transfer equilibrium. Rather, the initial diffusion rate in the first few minutes is measured. Thus, measurements can be made relatively quickly. Further, the diffusion chamber 115 can be incorporated in disposable small-scale bioreactors, such as T-flasks, cell culture bags, etc. With a predetermined sensor parameter (a), these types of disposable small-scale cell culture platforms can be put to immediate use for monitoring of DO₂ and DCO₂ in the culture.

The foregoing embodiments and advantages are merely exemplary, and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. Various changes may be made without departing from the spirit and scope of the invention, as defined in the following claims (after the Appendix below).

### APPENDIX

1. Bambot S B, Holavanahali R, Lackowicz J R, Carter J M, Rao G. Phase fluorometric sterilizable optical sensor. Biotechnol Bioeng. 1993; 43:1139-1145.
2. Kostov Y, Harms P, Randers-Eichhorn L, Rao G. Low-cost microbioreactor for high-throughput bioprocessing. Biotechnol Bioeng. 2001; 72(3):346-352.
3. Harms P, Kostov Y, Rao G. Bioprocess monitoring. Current Opinion in Biotechnology. 2002; 13:124-127.
4. Gupta A, Rao G. A study of oxygen transfer in shake flasks using a non-invasive oxygen sensor. Biotechnol Bioeng. 2003; 84(3):351-358.
5. Ge X, Kostov Y, Rao G. Low-cost noninvasive optical CO₂ sensing system for fermentation and cell culture. Biotechnol Bioeng. 2004; 89(3):329-334.
6. Betts J I, Baganz F. Miniature Bioreactors: Current Practices and Future Opportunities. Microbial Cell Factories. 2006; 5:21.
7. Chen A, Chitta R, Chang D, Anianullah A. Twenty-four Well Plate Miniature Bioreactor System as a Scale-Down Model for Cell Culture Process Development. Biotechnol Bioeng. 2009; 102(1):148-160.
8. Barrett T A, Wu A, Zhang H, Levy M S, Lye G J. Microwell Engineering Characterization for Mammalian Cell Culture Process Development. Biotechnol Bioeng. 2010; 105 (2):260-275.
9. Zanzotto A, Szita N, Boccazzi P, Lessard P, Sinskey A J, Jensen K F. Membrane-aerated microbioreactor for high-throughput bioprocessing. Biotechnol Bioeng. 2004; 87:243-254.
10. Kensy F, John G T, Hofmann B, Buechs J. Characterisation of operation conditions and online monitoring of physiological culture parameters in shaken 24-well microtiter plates. Bioprocess and Biosyst Eng. 2005; 28:75-81.
11. Zhang Z, Szita N, Boccazzi P, Sinskey A J, Jensen K F. A well-mixed, polymer-based microbioreactor with integrated optical measurements. Biotechnol Bioeng. 2006; 93:286-296.
12. Kirk T V, Szita N. Oxygen transfer characteristics of miniaturized bioreactor systems. Biotechnol Bioeng. 2013; 110:1005-1019.
13. Ge X, Hanson M, Shen H, Kostov Y, Brorson K A, Frey D D, Moreira A R, Rao G. Validation of an optical sensor-based high throughput bioreactor system for mammalian cell culture. J Biotechnol. 2006; 122:293-306.
14. Bambrick L L, Kostov Y, Rao G. In vitro cell culture pO₂ is significantly different from incubator pO₂. Biotechnol Prog. 2011; 27:1185-1189.

What is claimed is:

1. A system for measuring at least one analyte present in a medium, comprising:
a vessel adapted to a contain the medium;
a diffusion chamber positioned inside the vessel, wherein the diffusion chamber comprises at least one wall that defines a diffusion chamber volume and that is at least partially permeable to the at least one analyte;
an analyte sensing system coupled to the diffusion chamber for detecting portions of the at least one analyte that diffuse into the diffusion chamber from the medium; and
a controller operatively coupled to the analyte sensing system that determines initial rates of diffusion of the at least one analyte from the medium into the diffusion chamber based on amounts of the at least one analyte detected by the analyte sensing system, and for further determining a concentration of the at least one analyte in the medium based on the initial rates of diffusion.

2. The system of claim 1, wherein the medium comprises a cell culture.

3. The system of claim 2, wherein the at least one analyte comprises O₂ and/or CO₂.

4. The system of claim 3, wherein the analyte sensing system comprises an O₂ analyzer and/or a CO₂ analyzer.

5. The system of claim 2, wherein the vessel comprises a T-flask or a spinner-flask.

6. The system of claim 1, wherein the diffusion chamber comprises silicon tubing.

7. The system of claim 1, wherein the diffusion chamber and analyte sensing system are pneumatically coupled to at least one pneumatic pump.

8. The system of claim 7, wherein the diffusion chamber, analyte sensing system and pneumatic pump are pneumatically coupled with conduits and together form a circulation loop.

9. The system of claim 8, further comprising a valve pneumatically coupled to the circulation loop for selectively introducing nitrogen into the circulation loop for flushing the at least one analyte out of the diffusion chamber and analyte sensing system prior to initiating a measurement.

10. A system for measuring O₂ and CO₂ in a cell culture, comprising:
a vessel adapted to a contain the cell culture;
a diffusion chamber positioned inside the vessel, wherein the diffusion chamber comprises at least one wall that defines a diffusion chamber volume and that is at least partially permeable to O₂ and CO₂;
an O₂ analyzer and a CO₂ analyzer pneumatically coupled to the diffusion chamber for detecting O₂ and CO₂ that diffuse into the diffusion chamber from the cell culture; and
a controller operatively coupled to the O₂ analyzer and CO₂ analyzer that determines initial rates of diffusion of O₂ and CO₂ from the cell culture into the diffusion chamber volume based on the O₂ and CO₂ detected by the O₂ analyzer and CO₂ analyzer, and for further determining concentrations of O₂ and CO₂ in the cell culture based on the initial rates of diffusion.

11. The system of claim 10, wherein the vessel comprises a T-flask or a spinner-flask.

12. The system of claim 10, wherein the diffusion chamber comprises silicon tubing.

13. The system of claim 10, wherein the diffusion chamber, O₂ analyzer and CO₂ analyzer are pneumatically coupled to at least one pneumatic pump.

14. The system of claim 13, wherein the diffusion chamber, O₂ analyzer, CO₂ analyzer and pneumatic pump are pneumatically coupled with conduits and together form a circulation loop.

15. The system of claim 14, further comprising a valve pneumatically coupled to the circulation loop for selectively introducing nitrogen into the circulation loop for flushing O₂ and CO₂ out of the diffusion chamber, O₂ analyzer and CO₂ analyzer prior to initiating a measurement.

16. A method for measuring at least one analyte present in a medium, comprising:
positioning a diffusion chamber within the medium, wherein the diffusion chamber comprises at least one wall that defines a diffusion chamber volume and that is at least partially permeable to the at least one analyte;
flushing the diffusion chamber to substantially remove the at least one analyte from the diffusion chamber;
allowing the at least one analyte to diffuse from the medium into the diffusion chamber;
detecting portions of the at least one analyte that have diffused into the diffusion chamber;
determining initial rates of diffusion of the at least one analyte from the medium into the diffusion chamber based on the detected portions of the at least one analyte; and
determining a concentration of the at least one analyte in the medium based on the determined initial rates of diffusion.

17. The method of claim 16, wherein the medium comprises a cell culture.

18. The method of claim 17, wherein the at least one analyte comprises O₂ and CO₂.

19. The method of claim 16, wherein the diffusion chamber comprises silicon tubing.

20. The method of claim 18, wherein the diffusion chamber is adapted so that an amount of O₂ that diffuses into the diffusion chamber is sufficiently low as to not affect cell growth.

21. A probe system for measuring at least one analyte present in a medium, comprising:
a housing defined by housing walls that are impermeably to the analyte being measured;
a predetermined length of tubing that is permeable to the analyte being measured contained in the housing, wherein two open ends of the tubing extend through openings in a first end of the housing so as to be positioned outside the housing, and a sampling portion of the tubing extends through openings in a second end of the housing so as to be positioned outside the housing;
wherein the two open ends of the tubing are coupled to an analyte sensing system for detecting portions of the at least one analyte that diffuse into the sampling portion of the tubing from the medium when the sampling portion of the tubing is in contact with the medium; and
a controller operatively coupled to the analyte sensing system that determines initial rates of diffusion of the at least one analyte from the medium into the sampling portion of the tubing based on amounts of the at least one analyte detected by the analyte sensing system, and for further determining a concentration of the at least one analyte in the medium based on the initial rates of diffusion.

22. The probe system of claim 21, wherein the tubing comprises silicone tubing.

23. The probe system of claim 21, wherein a length of the sampling portion of the tubing can be selectively increased by pulling additional tubing out of the housing through the openings in the second end of the housing, and can be selectively decreased by pushing a portion of the sampling tubing into the housing through the openings in the second end of the housing.

24. The probe system of claim 21, wherein the housing is adapted to be at least partially inserted into soil for measuring analytes in the soil.

25. The probe system of claim 21, wherein the housing is adapted to be at least partially inserted into a liquid for measuring analytes in the liquid.

26. The probe system of claim 21, wherein the housing is adapted to be at least partially inserted into a gas environment for measuring analytes in the gas environment.

27. The probe system of claim 21, wherein the housing is adapted to be at least partially inserted into a human body for measuring analytes in the human body.

28. A probe for measuring at least one analyte present in a medium, comprising:
a housing defined by housing walls that are impermeably to the analyte being measured; and
a predetermined length of tubing that is permeable to the analyte being measured contained in the housing, wherein two open ends of the tubing extend through openings in a first end of the housing so as to be positioned outside the housing, and a sampling portion of the tubing extends through openings in a second end of the housing so as to be positioned outside the housing;
wherein the two open ends of the tubing are adapted to be coupled to an analyte sensing system for detecting portions of the at least one analyte that diffuse into the sampling portion of the tubing from the medium when the sampling portion of the tubing is in contact with the medium.

29. The probe of claim 28, wherein the tubing comprises silicone tubing.

30. The probe of claim 28, wherein a length of the sampling portion of the tubing can be selectively increased by pulling additional tubing out of the housing through the openings in the second end of the housing, and can be selectively decreased by pushing a portion of the sampling tubing into the housing through the openings in the second end of the housing.

31. The probe of claim 28, wherein the housing is adapted to be at least partially inserted into soil for measuring analytes in the soil.

32. The probe of claim 28, wherein the housing is adapted to be at least partially inserted into a liquid for measuring analytes in the liquid.

33. The probe of claim 28, wherein the housing is adapted to be at least partially inserted into a gas environment for measuring analytes in the gas environment.

34. The probe of claim 28, wherein the housing is adapted to be at least partially inserted into a human body for measuring analytes in the human body.
